(19) United States
(12) Patent Application Publication
(10) Pub. No.: US 2011/0276106 A1
(43) Pub. Date: Nov. 10, 2011

(54) MENSTRUATION PAIN RELIEF DEVICE

(76) Inventor: CHIEN-CHOU CHEN, TAINAN COUNTY (TW)

(21) Appl. No.: 12/776,581

(22) Filed: May 10, 2010

(51) Int. Cl. A61N 1/00

(52) U.S. Cl. 607/46

(57) ABSTRACT

The present invention relates to a menstruation pain relieve device, which comprises a female brief, an electrothermal sheet and a power supply. The most important advantage is that this invention jointed on the female brief can particularly ease off the dysmenorrheal occurred to females during menstrual periods. The electrothermal sheet is fitted on the brief where corresponds to the female's abdomen. A lead wire with a joint is connected to the electrothermal sheet at one end thereof and the power supply is connected to the joint of the lead wire by means of a connection end, so that power can be supplied to the electrothermal sheet to generate proper amount of heat and then further transmit the same to the female's abdomen for achievement of easing off dysmenorrhea occurred during menstrual periods.

### BACKGROUND OF THE INVENTION

1. Fields of the Invention

The present invention relates to a pain relief device, especially to a menstruation pain relief device in which an electrothermal device for generating heat is mounted in female underwear so as to alleviate discomfort and menstruation pain during periods.

2. Descriptions of Related Art

Menstruation pain (dysmenorrhea) is the most common gynecologic problem. Dysmenorrhea affects more than 60 percent of all women who have a menstrual period. For about 10 percent of these women, menstrual pain is a common cause of absenteeism at school and work. There are two types of dysmenorrhea: primary dysmenorrhea and secondary dysmenorrhea. Primary dysmenorrhea is common menstrual cramps that usually begin one to two years after a woman starts getting her period in the absence of pelvic pathology. Pain occurs just the first two days of menstruation. Secondary dysmenorrhea is pain that is caused by a disorder in the woman's reproductive organs, starting several years after the first period. Symptoms and degree of pain continue or get worse. And Pain from secondary dysmenorrhea usually begins earlier in the menstrual cycle and lasts longer than common menstrual cramps.

Pain from primary dysmenorrheal usually starts several hours before menstrual flow or accompanying menstruation, continuing through several hours to several days. Pain is usually felt in the lower abdomen. Some people have other symptoms such as nausea, vomiting, fatigue, diarrhea, backache, headache, etc. Release of prostaglandins and other inflammatory mediators in the uterus cause the uterus to contract. The uterus contracts strongly and they constrict the blood supply to the uterine tissue. Pain or cramps results when part of the uterine tissue loses its supply of oxygen. Beside pain in lower abdomen, the muscular cramps even cause lower back pain or upper thigh pain.

Secondary dysmenorrhea is dysmenorrhea which is associated with pelvic lesions. The possible causes of secondary dysmenorrhea include endometriosis, pelvic congestion syndrome, pelvic inflammatory disease, uterine fibroid or ovarian cyst, endometrial polyp, intrauterine devices, intrauterine adhesion or uterine abnormality. These patients require detailed interview, gynecological examinations and laparoscopy to find out the cause and suit the remedy to the case. The most common cause of secondary dysmenorrhea is endometriosis. One third to one half of these women suffering from menstrual pain during their period have endometriosis.

Therapies for mild menstrual pain consists of resting for a whole day, heating pad applied to the lower abdomen, hot drinks, a hot bath, and massage the abdomen so as to increase the pelvic blood circulation and ease the pain. If the pain gets worse, some women take pain relief medicines. Yet this is not a good choice because they may need more pills and have effects on their health.

Thus there is a need to provide a menstruation pain relief device with novel design and higher practical value that overcomes above shortcomings.

### SUMMARY OF THE INVENTION

Therefore it is a primary object of the present invention to provide a menstruation pain relief device that helps women to ease the pain they experience during menstruation. The pain is relieved by applying heat generated from an electrothermal device mounted in women's underwear, near the abdomen.

In order to achieve above object, a menstruation pain relief device of the present invention includes an electrothermal device mounted in the female brief, corresponding to lower abdominal area of the female brief and generating heat so as to alleviate discomfort and pain during the period. A lead wire with a joint is connected with the electrothermal sheet and a power supply is connected to the joint. Thus power is supplied to the electrothermal sheet and heat is generated. Thereby heat energy is transferred from the electrothermal sheet to women's abdomen for relieving discomfort and menstruation pain during periods.

Moreover, the electrothermal sheet is connected to and located on the lower abdominal area of the female brief by a piece of cloth sewn on the female brief.

Furthermore, the joint is a socket that is assembled and electrically connected with a plug on the connection end of the power supply. Or the joint is a plug that is assembled and electrically connected with an insertion hole on the connection end of the power supply.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is an explosive view of an embodiment according to the present invention;

FIG. 2 is a schematic drawing showing an assembled embodiment according to the present invention;

FIG. 3 is a schematic drawing showing another embodiment according to the present invention.

### DETAILED DESCRIPTION OF THE PREFERRED EMBODIMENT

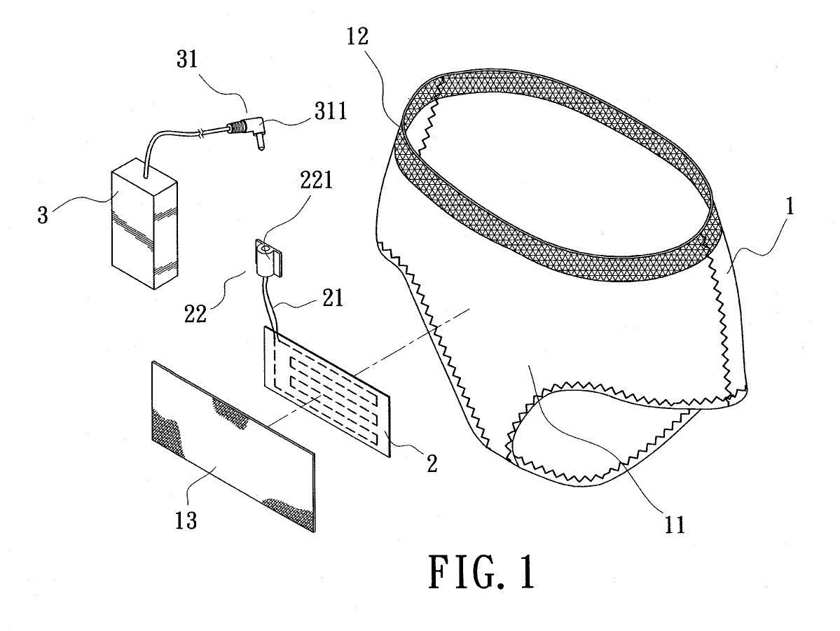

Refer to FIG. 1, a menstruation pain relieve device of the present invention is connected to a female brief 1. The menstruation pain relieve device includes an electrothermal sheet 2 arranged corresponding to lower abdominal area 11 of the female brief 1, a lead wire 21 with a joint 22 connected with the electrothermal sheet 2, and a power supply 3. The joint 22 is positioned on an elastic band 12 of the female brief 1 and is electrically connected with a connection end 31 of the power supply 3 so as to make the electrothermal sheet 2 generate heat.

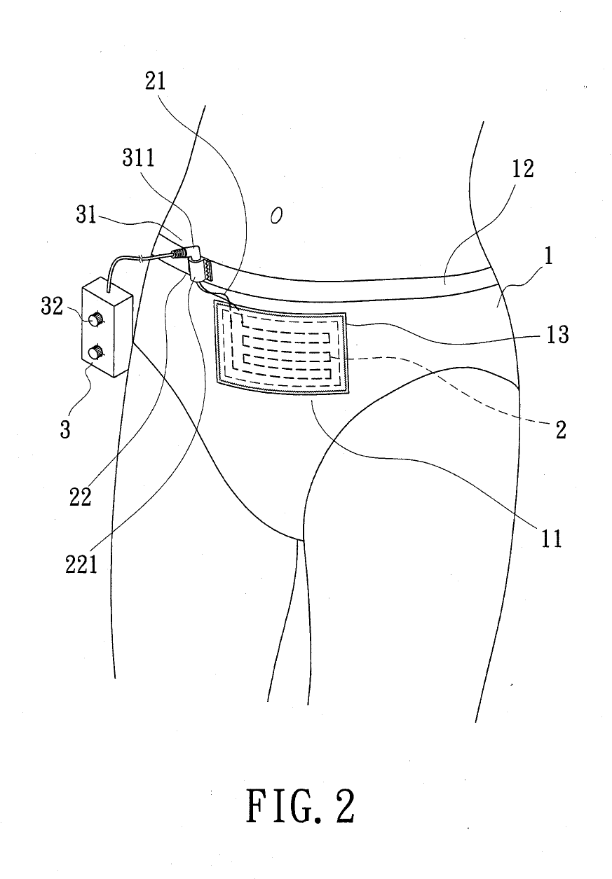

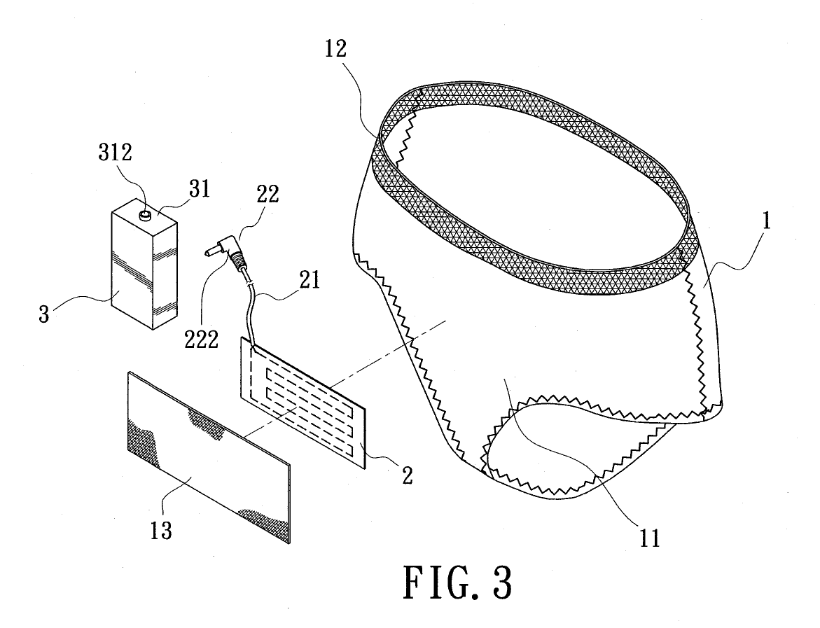

Refer from FIG. 1 to FIG. 3, while in use, the electrothermal sheet 2 is connected with the female brief 1 by sewing a piece of cloth 13 onto the lower abdominal area 11 of the female brief 1. Then the joint 22 on one end of the lead wire 21 of the electrothermal sheet 2 is fixed on the elastic band 12 of the female brief 1 correspondingly. The joint 22 is attached by sewing or adhesion. The joint 22 is a socket 221 while the connection end 31 of the power supply 3 is a plug 311. The plug 311 is inserted into the socket 221 so that the power supply 3 and the electrothermal sheet 2 are electrically connected with each other, as shown in FIG. 1 and FIG. 2. Or the joint 22 can be a plug 222 that is electrically connected with an insertion hole 312 on the connection end 31 of the power supply 3 correspondingly, as shown in FIG. 3. After electrical connection has been made, the electrothermal sheet 2 generates heat that is transferred to women's abdomen so as to reduce the pain and discomfort during the period.

In addition, the power supply 3 is set with a temperature control switch and a time switch 32. The power supply 3 is mounted in users' pockets or is hung on user's cloth that is arranged with a hook correspondingly.

Compared with menstruation pain relief devices available now, the present device has following advantages:
1. By means of the electrothermal sheet that is disposed on underwear for generating heat, the menstruation pain relief device of the present invention helps to ease menstruation pain.
2. The menstruation pain relief device of the present invention is not only easy to set up but also convenient to use.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A menstruation pain relief device that is connected to a female brief comprising:
an electrothermal sheet arranged corresponding to lower abdomen area of the female brief;
a power supply;
a lead wire with a joint connected with the electrothermal sheet; the joint positioned on an elastic band of the female brief and electrically connected with a connection end of the power supply so as to make the electrothermal sheet generate heat.

2. The device as claimed in claim 1, wherein the electrothermal sheet is located and connected to the female brief by being sewn together with a piece of cloth to the lower abdomen area of the female brief.

3. The device as claimed in claim 1, wherein the joint is a socket that is assembled and electrically connected with a plug on the connection end of the power supply.

4. The device as claimed in claim 1, wherein the joint is a plug that is assembled and electrically connected with an insertion hole on the connection end of the power supply.

5. The device as claimed in claim 1, wherein the power supply is disposed with a temperature control switch and a time switch.
